(19) United States
(12) Patent Application Publication
(10) Pub. No.: US 2004/0019433 A1
(43) Pub. Date: Jan. 29, 2004

(54) Method for locating areas of interest of a substrate

(76) Inventor: Wilhelmus Marinus Carpaij, Liempde (NL)

Correspondence Address: Kenneth P George, Amster Rothstein & Ebenstein, 90 Park Avenue, New York, NY, 10016, US

(21) Appl. No.: 10/333,365

(22) PCT Filed: Jul. 11, 2001
(86) PCT No.: PCT/EP01/08012

(30) Foreign Application Priority Data
Jul. 18, 2000 (EP) 00202551.8

(51) Int. Cl. G06F 19/00; G01N 33/48; G01N 33/50; G06K 9/00

(52) U.S. Cl. 702/20; 382/128

(57) ABSTRACT

In a method for evaluating the signal intensity of at least one area of a substrate embedded in a substrate surrounding with a background intensity by means of a computer, a scatter parameter of the substrate is determined. Further, a matrix (IS) of pixel intensities of pixels located within an evaluation window enclosing the area and surrounding is determined. The pixel intensities of the matrix (IS) are used to obtain a histogram of pixel intensities. This evaluation histogram shows two distribution peaks, the first distribution peak with the lowest intensity corresponding with the surrounding pixels and the second distribution peak with the highest intensity corresponding with the area pixels. A curve with two peaks is fitted on this evaluation histogram. The scatter parameter is used to correct for scattering either the matrix (IS) or the fitted curve. The signal intensity of pixels in the area is determined by means of data obtained from the curve fitted on tile pixel intensity histogram.

The present invention relates to a method for evaluating the signal intensity of at least one area of a substrate embedded in a substrate surrounding with background intensity by means of a computer, and to a method for locating possible areas of interest of a substrate embedded in a substrate surrounding.

U.S. Pat. No. 5,795,716 discloses a method of this type which is used in a computer-aided visualisation and analysis system for sequence evaluation. Generally the substrate comprises an array of areas, each area having a known binding substance or probe, capable of specifically binding with an analyte. Assays in which an array can be used may include sequencing by hybridization, immunoassays, receptor/ligand assays etc. For example, the array may be used to screen a biological sample, such as blood for the presence of a large number of analytes. If the substrate is brought into contact with a liquid that contains one or more analytes, a reaction pattern may occur representing the specific affinity of the analytes(s) for the binding substances of the array. The array may consist of areas comprising nucleic acid probes. The array may be used for the detection and/or typing of viral or bacterial nucleic acid or for mutation detection. Likewise an array can be used for performing immunoassays. In that case the binding substances or probes may be antigens (peptides) or antibodies. By binding of an analyte to the binding substance a detectable signal, such as a fluorescent signal, is generated, for example, by binding with a second, labeled reagent. For instance, a scanner generates an image file and this image file is evaluated to determine the signal intensity of each area. To obtain accurate information, it is very important to accurately determine the signal intensity of the area where a binding substance is located. In the known method, this signal intensity is determined using a background intensity of a “blank” area, wherein is it assumed that the blank area provides a background intensity only. However, it is very difficult to find an area on the substrate providing with certainty background intensity only. Further, in many of the known methods one background intensity determined in this manner is used for all areas so that any variations in background intensity along the surface of the substrate are not considered. Moreover, any effects on the signal intensity measured caused by scattering in the substrate is not considered at all.

The invention aims to provide an improved method of the above-mentioned type.

According to the invention a method for evaluating the signal intensity of at least one area of a substrate embedded in a substrate surrounding with a background intensity by means of a computer, is characterized in that a scatter parameter of the substrate is determined, and in that a matrix (I_{S}) of pixel intensities of pixels located within an evaluation window enclosing the area and surrounding is determined, wherein the pixel intensities of the matrix (I_{S}) are used to obtain an evaluation histogram of pixel intensities, said evaluation histogram showing two distribution peaks, the first distribution peak with the lowest intensity corresponding with the surrounding pixels and the second distribution peak with the highest intensity corresponding with the area pixels, wherein a curve with two peaks is fitted on said evaluation histogram, wherein the scatter parameter is used to correct for scattering either the matrix (I_{S}) or the curve, wherein the signal intensity of pixels in the area is determined by means of data obtained from the curve fitted on said evaluation histogram.

In this manner a method is obtained, wherein the mean signal intensity of pixels in the area is determined with increased accuracy by correcting the pixel intensities of both the area and surrounding for the scatter characteristics of the substrate. In the method of the invention evaluation of the background intensity is an inherent part of the evaluation of the signal intensity of the area of interest. Thereby separate steps for locating a part of the substrate for determining the background intensity are not required and, moreover, the background intensity of each area is determined at a location close to the corresponding area so that variations in background intensity along the surface of the substrate do not affect the evaluation result.

The invention further provides a method for locating possible areas of interest of a substrate embedded in a substrate surrounding by means of a computer, characterized in that an image file of the substrate is processed in a low pass filter algorithm to determine a matrix of local mean values for all pixels of the image file, wherein the matrix of local mean values is combined with the matrix of actual pixel values of the image file to obtain a first matrix of high/low pixel values which are either high or low depending on the actual pixel value being above or below the corresponding local mean value, wherein the high/low pixel values of the first matrix are further processed in a median filter algorithm to obtain a second matrix of median pixel values, wherein each median pixel value equals the majority of the high/low pixel values of the first matrix, wherein the median pixel values are processed row by row and column by column to determine the mean row values and mean column values, respectively, wherein the rows and columns with the highest mean row values and highest mean column values are selected as estimates of the row centre lines and column centre lines of possible areas of interest, the intersections of which are the centres of possible areas of interest.

The invention will be further explained by reference to the drawings showing some diagrams to illustrate two embodiments of the method of the invention.

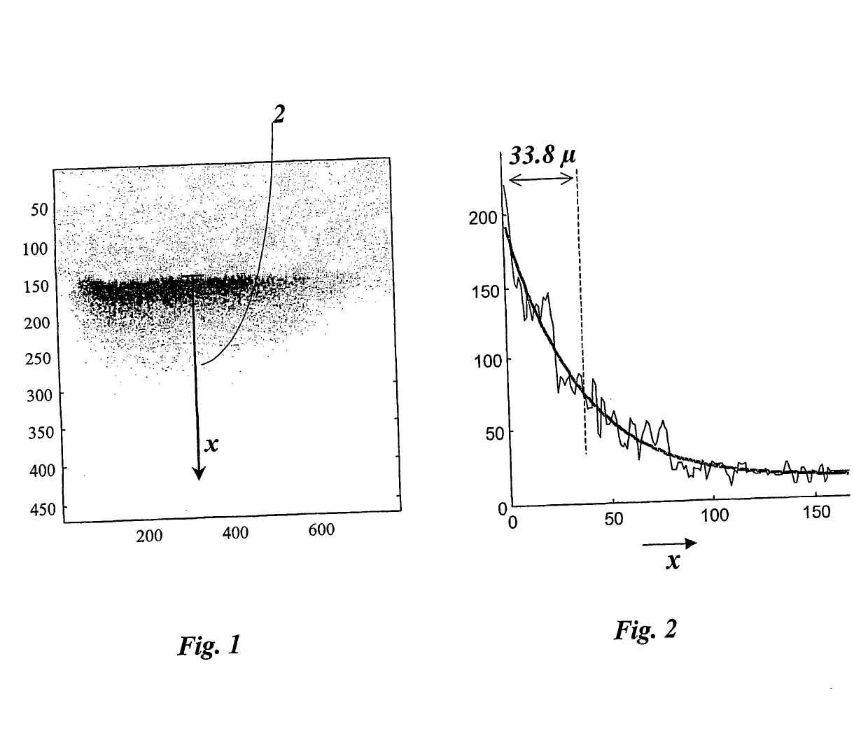

FIG. 1 shows an image used to determine a scatter parameter of the substrate.

FIG. 2 shows the scatter decay function obtained form the image of FIG. 1.

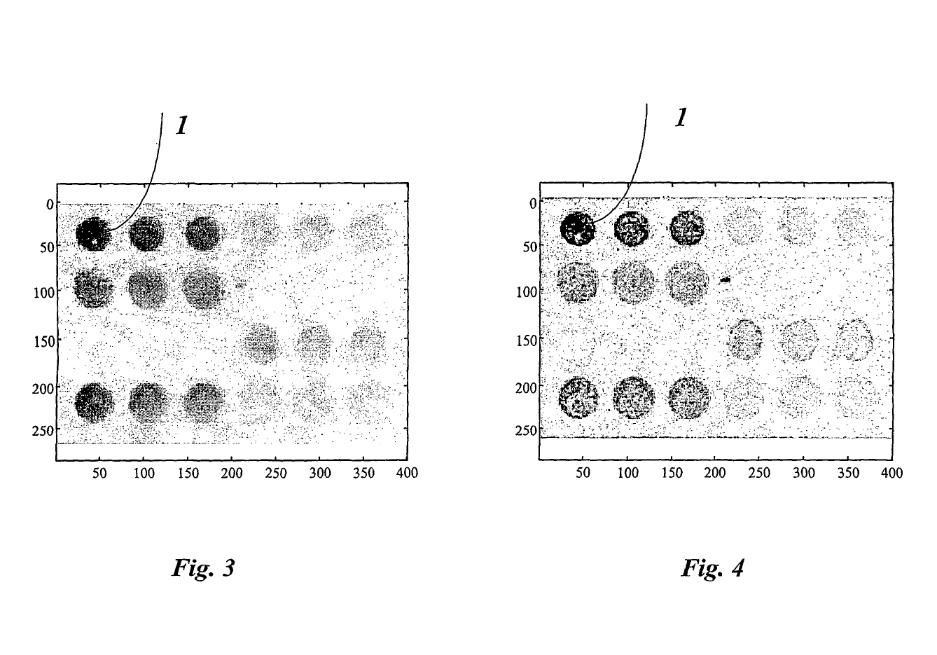

FIG. 3 shows an image of a substrate with an array of 6×4 areas, each area having a known probe and after deposition of a labelled material on the substrate.

FIG. 4 shows the image of FIG. 3 after mathematically removing the scatter effects.

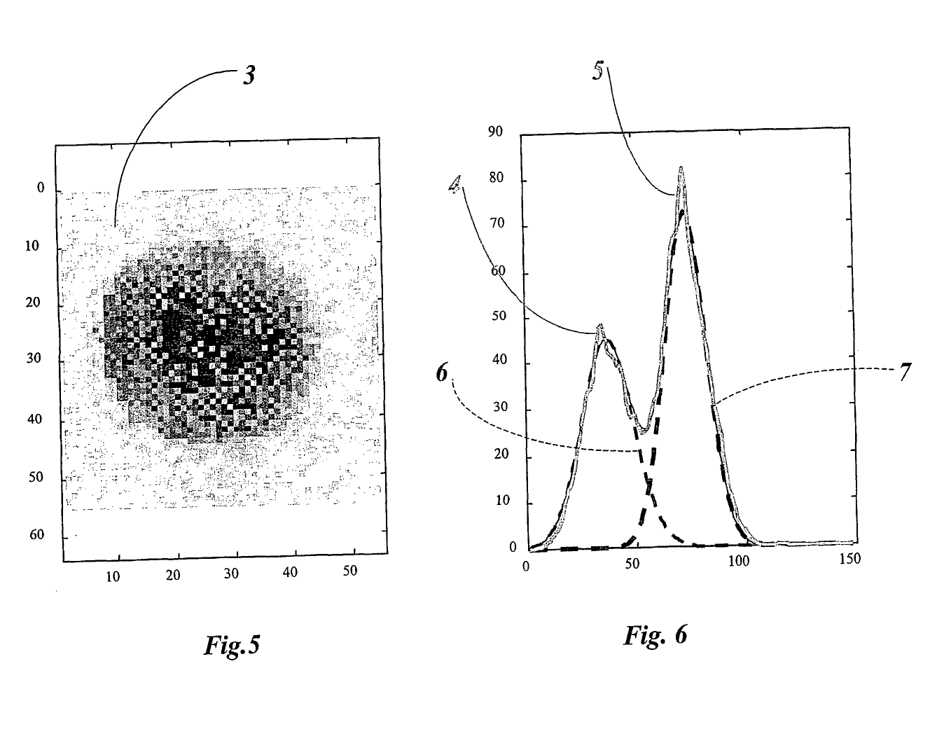

FIG. 5 shows an evaluation window on one of the areas of FIG. 4.

FIG. 6 shows the evaluation histogram of a matrix of deconvoluted pixel intensities together with two fitted curves.

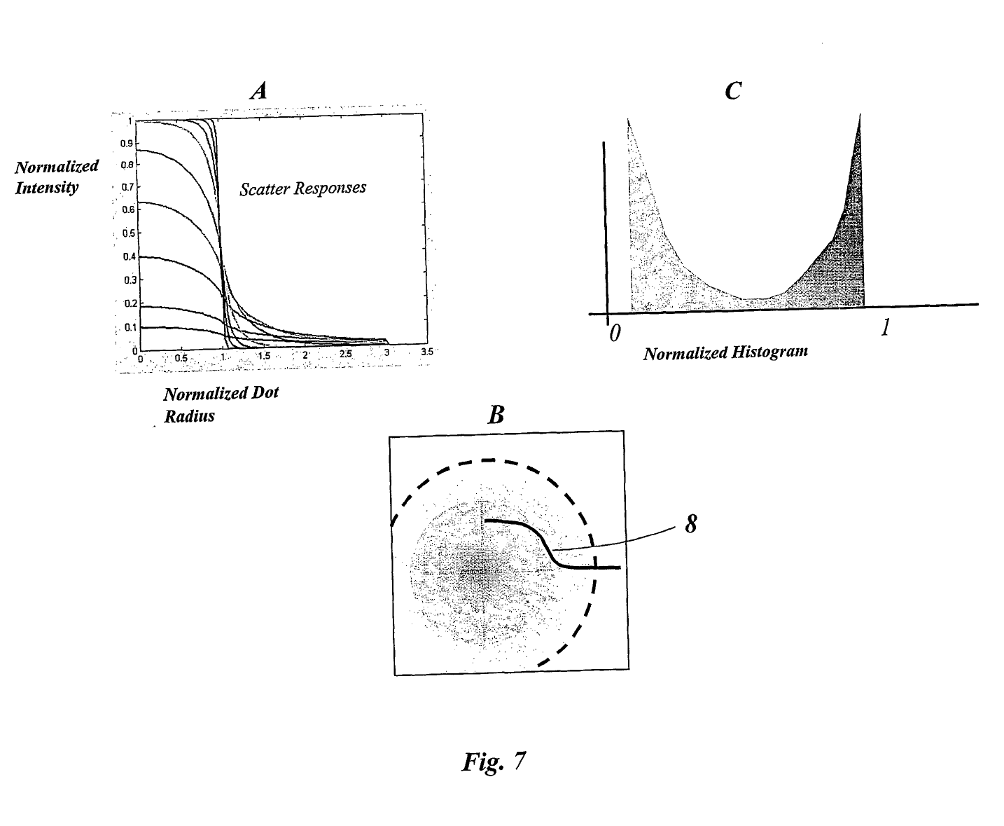

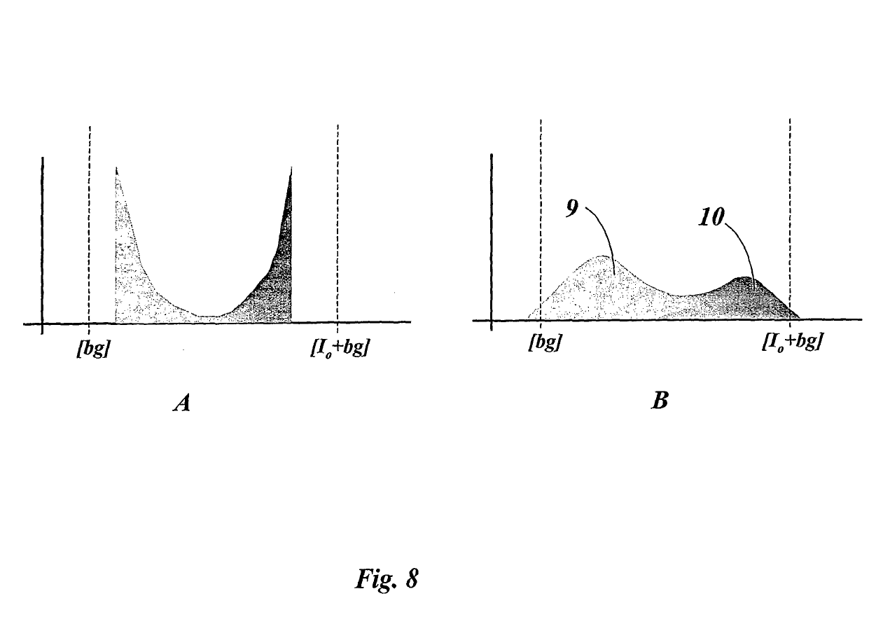

FIGS. 7 and 8 illustrate the determination of a theoretical scatter histogram used for fitting in a second embodiment of the method of the invention.

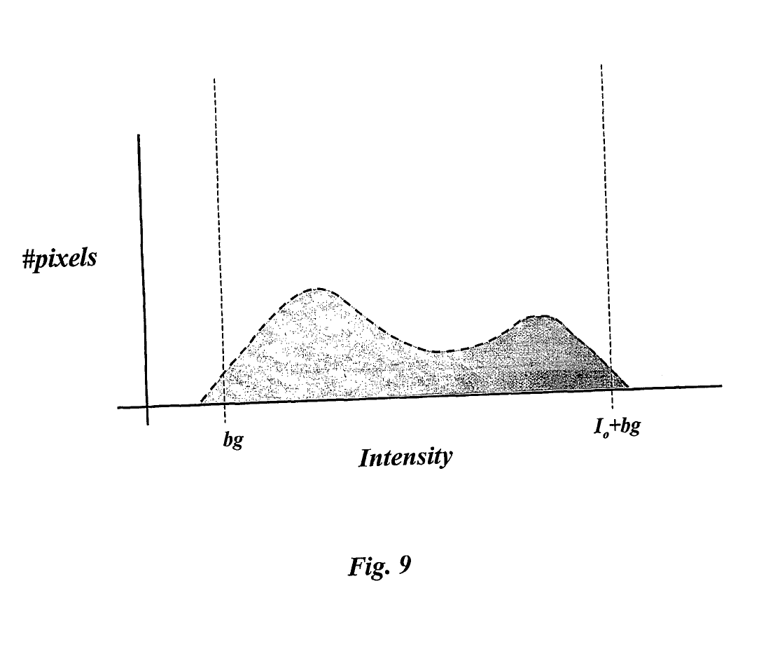

FIG. 9 shows the evaluation histogram obtained from the pixel intensities of the matrix I_{S} of an area of FIG. 3 together with a fitted theoretical histogram obtained in the manner as shown in FIGS. 7 and 8.

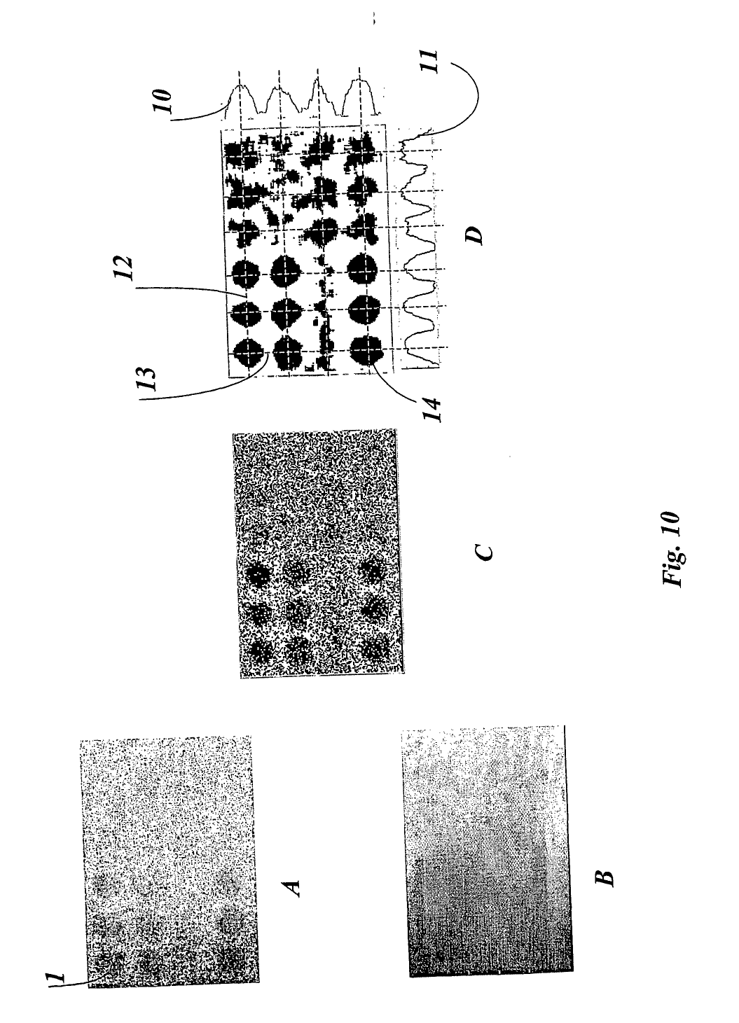

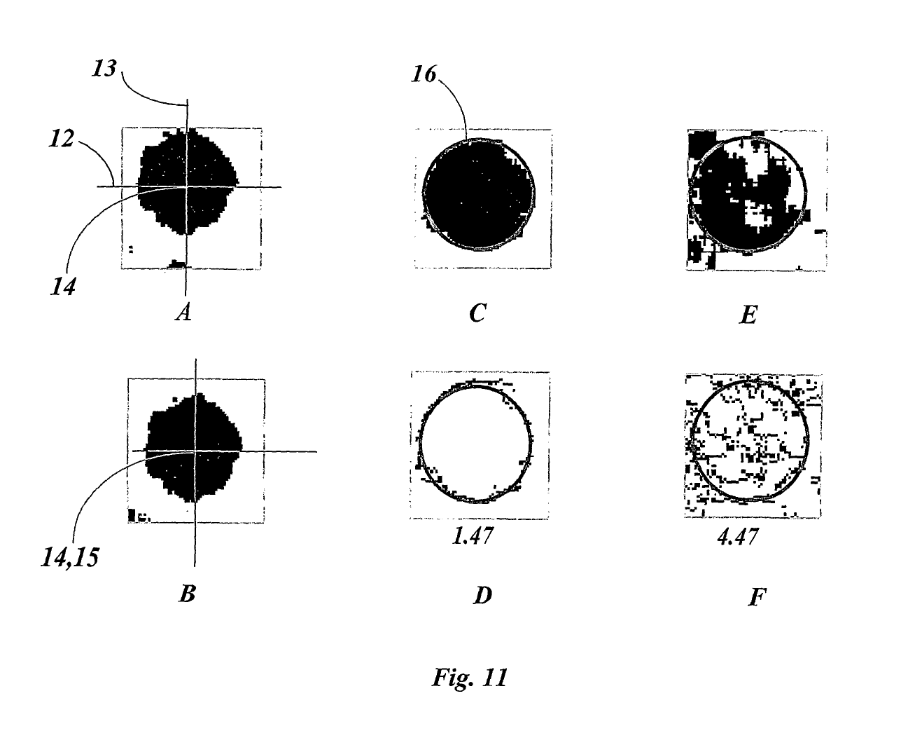

FIGS. 10A-10D and 11A-11F show steps in a preferred embodiment of a method of the invention to evaluate an image file of a substrate as shown in FIG. 3 to locate possible areas of interest.

The method of the invention for evaluating the signal intensity of an area of a substrate embedded in a substrate surrounding is implemented as a computer program for use in a computer. The method can be used for example for sequence evaluation by analysing the signal intensities of hybridised nucleic acid probes. However, as mentioned above the substrate can be used for any other assay, such as immunoassays, receptor/ligand assays etc. As the present method only relates to evaluating the signal intensity, the manner for sequence evaluation will not be described in further detail. Reference is made to WO 9902266 for example.

In such a sequence evaluation, a substrate as described in WO 9902266 can be used for example, having in the case shown in FIG. 3 an array of 6×4 areas 1, each area or dot having a probe with a known sequence. When a material with a fluorescence label is contacted with the surface of the substrate, different concentrations of the labelled material will be found in the different dot areas. Illumination of the substrate will yield fluorescence light originating from each dot area 1 and FIG. 3 shows an image obtained by means of a CCD camera. It is noted that although in this case fluorescence light is obtained, the method is not restricted to evaluating fluorescence intensities. The method encompasses evaluating any type of signal intensity originating from an area embedded in a surrounding. The image as shown in FIG. 3 is stored in the computer, for example on hard disk, as an image file. As can be seen in FIG. 3, there is no sharp transition from a dot area 1 to its surrounding so that it is difficult to find a location for determining the background intensity in the vicinity of a dot area 1. The gradual or blurred transition from dot area to surrounding is caused by scattering of the fluorescent light of the labelled material. In order to correct for this scattering effect, a scatter parameter of the substrate is determined in the method of the invention.

FIG. 1 shows by way of example a manner of determining a scatter parameter of the substrate. FIG. 1 is an image of a sidewise illuminated substrate having a thickness of 60μ in this example. In FIG. 1 a line 2 is shown along which the pixel intensities are determined. It is noted that the wording “pixel intensity” is used to indicate the signal intensity obtained from one pixel of the CCD camera or any other type of imaging device used to provide the image of the array. From the pixel intensities measured along this line 2 an exponential scatter function can be made as shown in the diagram of FIG. 2 having in this case a characteristic decay of 33,8μ. This characteristic scatter decay is used in the method of the invention to correct the pixel intensities measured for the scatter effects of the substrate. As the scatter decay determined in this manner is measured by sidewise illumination of the substrate, any illumination not scattered and going directly towards the imaging device used in normal measuring circumstances is not taken into account. To correct for such signal intensity originating directly from the substrate without scattering a predetermined percentage of the received signal intensity for all pixels can be deducted for example.

It is noted that in FIG. 1 a dry substrate is measured. Of course, scatter parameters of the same substrate for a wet state can be determined in a corresponding manner. Further scatter parameters for different types of substrates can be determined and stored in the computer. In the evaluation method, a stored scatter parameter can be selected from the memory of the computer in accordance with the type and state of the substrate used. It is also possible to enter a known scatter parameter of the substrate used through a suitable input device. In this respect it is noted that the step of determining a scatter parameter of the substrate encompasses any manner of input of a previously determined scatter parameter for use in the method described.

The dot areas 1 are evaluated dot by dot. For this evaluation one dot area 1 is isolated with its direct surrounding from the remainder of the substrate image by means of an evaluation window 3 which is shown in FIG. 5. The size of the evaluation window 3 is such that the complete dot area 1 and its direct surroundings are located within the evaluation window 3. In the example of FIG. 5, the window 3 comprises 55×55 pixels. In isolating the dot areas 1, the computer program can first evaluate the complete image of FIG. 3 to locate the centre of each dot area 1 and to determine the size of the evaluation window 3 to be used. By means of a suitable user interface the location of the centre of each dot area and/or size of the window 3 can be changed. A favourable manner of evaluating the complete image of FIG. 3 to locate the dot centres will be described hereinafter.

The intensities of all pixels within the evaluation window 3 are inserted in the matrix I_{S} of pixel intensities, i.e. these intensities are representing signal intensities including the scatter effects of the substrate. These pixel intensities will be referred to as scattered pixel intensities in this description. As the scatter parameter of the substrate is known, the scatter effect can be mathematically corrected. In the first embodiment of the method described, the scatter effect is taken into account by a deconvolution. Deconvolution methods as such are known in mathematics. The result of deconvoluting the matrix I_{S} of scattered pixel intensities with the scatter parameter of the substrate is a matrix ID of non-scattered pixel intensities, i.e. these non-scattered pixel intensities are representing signal intensities corrected for scatter effects. FIG. 4 shows the display on the computer monitor of the image file of FIG. 3 after deconvolution. In the display of this processed image file it can be seen that the blurring effect of scattering at the transition of dot area and its direct surroundings is removed.

In a further step of this embodiment, the non-scattered pixel intensities of the matrix I_{D} are used to determine a pixel intensity evaluation histogram, which evaluation histogram is shown in FIG. 6. Clearly, this histogram of pixel intensities shows two distribution peaks 4 and 5, wherein the first distribution peak 4 with the lowest intensity represents the surrounding pixels and the second distribution peak 5 with the highest intensity represents the pixels of the dot area 1.

As both these distributions 4 and 5 will generally resemble normal distributions, a standard fitting method can be used to fit two normal distribution curves 6 and 7 on the distribution peaks 4 and 5. As such fitting methods are known per se, a detailed description is deemed to be superfluous. For example a least mean square method can be used to find the best fitting curves. In determining the normal distribution curves 6 and 7 a noise parameter of the noise present in the pixel intensities of the matrix (I_{S}) or in all pixel intensities of the complete image file of the substrate of FIG. 3 is used, in particular the standard deviation of this noise. This standard deviation σₙₒᵢₛₑ is determined by taking for example the difference between adjacent pixels resulting in a distribution with a mean value 0 and a standard deviation a σ=σₙₒᵢₛₑ*√2.

Finally, the mean value of the first fitted distribution curve 6 is taken as the best estimate for the mean pixel intensity of the pure background intensity. The mean value of the second fitted distribution curve 7 is taken as the best estimate of the mean pixel intensity of the dot area 1, i.e. background intensity together with fluorescence intensity of the labelled material. Therefore, the difference of both mean values provides the mean fluorescence intensity of the pixels in the dot area 1, i.e. of the labelled material.

In a second embodiment of the method of the invention, the scatter parameter of the substrate is taken into account in a different manner. For, it is known that deconvolution amplifies the noise present in the data obtained from the CCD camera. In case of low signal intensity signals, evaluation by deconvolution yields poor results.

In accordance with the second embodiment of the invention, the scatter parameter is taken into account by determining the scatter response of a predetermined theoretical label area as schematically shown in FIGS. 7A-7C. In FIG. 7A different scatter responses are shown for different scatter parameters, i.e. scatter parameters for different substrates and different substrate conditions. The straight line at x=1 corresponds with the non-scattered response curve, i.e. an intensity I within the theoretical label area and an intensity zero in the surrounding of the area. Combining this non-scattered response with the different scatter parameters results in the different scatter responses shown in FIG. 7A. It is noted that in FIG. 7A the scatter responses are shown for normalized label area and normalized intensity, i.e. with the radius R of the area and the intensity I as units. FIG. 7B shows an evaluation window of the same size as the evaluation window used in FIG. 5 showing the scattered response of the theoretical label area together with a corresponding scatter response curve 8. As this scatter response curve 8 shows the intensities along one radius from the centre of the theoretical area up to the edge of the evaluation window, a normalized histogram of pixel intensities as shown in FIG. 7C can be made by using the pixel intensity values provided by the scatter response curve 8 and the number of pixels surrounding the centre of the label area.

FIG. 8 shows two next steps in the second embodiment of the method of the invention. According to FIG. 8A the normalized histogram of FIG. 7C is scaled by choosing a specific background intensity I_{B} and area intensity I_{A} and this scaled histogram is convoluted with the noise distribution parameter obtained in the above-described manner. In this manner a theoretical scatter histogram is obtained and this theoretical scatter histogram shows two distribution peaks 9 and 10 representing the background intensity caused by scattering and the area intensity. The theoretical scatter histogram is fitted on an evaluation histogram obtained from the scattered pixel intensities of the matrix I_{S}. Standard fitting methods can be used to find the best fitting theoretical scatter histogram by varying at least the background intensity I_{B} and area I_{A} intensity values. As a further alternative it is possible to also vary the radius value of the theoretical area, the standard deviation of the noise and the scatter parameter value used.

FIG. 9 shows the evaluation histogram obtained from the scattered pixel intensities of the matrix I_{S} with the best fitting theoretical scatter histogram. When the best fitting theoretical scatter histogram curve is found, the corresponding background intensity and area pixel intensity values are used as best estimates for the background intensity and area pixel intensity. The difference of area pixel intensity and background intensity corresponds with the fluorescence intensity of the pixels in the dot area 1.

It is noted that in the above examples of the method described only the mean value of the peaks of the fitted curve are used for evaluation. The fitted curve comprises further information which can be used for examination. For example, the surface area of each peak can be used to determine the number of pixels of a dot area and the surrounding background.

FIGS. 10A-10D and FIGS. 11A-11F show steps in a preferred embodiment of a method to evaluate an image file of a substrate as shown in FIG. 3 to locate the centres of dot areas 1 and to determine the size of the evaluation window 3 to be used in the above referenced methods. It is noted that this method of evaluating the complete image file can be used in combination with any method of evaluating one dot area and this method is therefore not restricted to the above described methods or any other specific dot area evaluation method.

FIG. 10A shows the display of an image file of a substrate corresponding to the substrate in FIG. 3. Although this image file clearly comprises noise, some possible areas of interest can be recognised as dot areas 1 in the display of the unprocessed pixel values of the pixels of the image file. For locating all possible areas of interest, the pixel values of the image file are processed in a low pass filter algorithm to determine a matrix of local mean values for all pixels of the image file. The thus obtained matrix of local mean values for all pixels of the image file is shown in FIG. 10B. These local mean values are obtained by using a second window not shown having a size preferably between 25-50% of the evaluation window used to evaluate the signal intensity of a dot area. This second window is slided along the pixels of the image file pixel by pixel. At each position of the second window the mean value of all pixels of the image file within the second window is taken as the local mean value of the pixel at the centre of the second window. By sliding the second window along the complete surface of the image file, a local mean value is determined for each pixel of the image file. In this manner actually all high frequency information of the image file is removed.

The matrix of the actual pixels values of the image file is combined with the matrix of local mean values obtained in the above described manner to obtain a first matrix of high/low pixel values for all pixels of the image file. These high/low pixels values are either high or low depending on whether the actual pixel value is above of below the corresponding local mean value of the pixel. This means that if an actual pixel value is above the corresponding local mean value, the high/low pixel value of the first matrix will be high, whereas if the actual pixel value is below the corresponding local mean value, the high/low pixel value of the first matrix will be low. The thus obtained first matrix is displayed in FIG. 10C.

The high/low pixel values of the first matrix are further processed in a median filter algorithm to obtain a second matrix of median pixel values for all pixels of the image file. The thus obtained matrix of median pixel values is shown in FIG. 10D. According to this median filter algorithm the high/low pixel value of each pixel of the first matrix is compared with the high/low pixel values of the pixels immediately surrounding this pixel. The high/low pixel value is made equal to the value of the majority of the high/low pixel values of these surrounding pixels and this new value is referred to as median pixel value.

As shown in FIG. 10D, the thus obtained matrix clearly shows all possible areas of interest. A first rough estimate for the location of the centres of these possible areas of interest is obtained by determining the mean row values and mean column values row by row and column by column, respectively. These mean row values and mean column values are determined by adding row by row and column by column all median pixels values of the second matrix and dividing the thus obtained value by the number of pixels. These mean values are shown at the bottom and right sides of the display of the image file in FIG. 10D as curves 10 and 11. As it is known that a substrate with 6×4 possible areas of interest are used, the four highest mean row values and six highest mean column values are selected as estimates of row centre lines 12 and column centre lines 13 of the possible areas of interest. The intersections of these row and column centre lines 12, 13 are used as the location of the centres 14 of all possible areas of interest.

FIGS. 11A-11F show further steps in the method to locate the centres of possible areas of interest. FIG. 11A shows an evaluation window on an area of interest with the row centre line 12 and the column centre line 13. In a further step it is determined whether the intersection 14 of lines 11, 12 is indeed in the centre of gravity 15 of the pixels of the area of interest. To this end the centre of gravity is determined from the median pixel values of the pixels of the second matrix within the evaluation window shown in FIG. 11A. If this centre of gravity does not coincide with the intersection 14, the centre lines 12, 13 are shifted such that their intersection coincides with the centre of gravity 15 as shown in FIG. 11B.

To decide whether the pixels within the evaluation window having a high value are indeed representing an area of interest or just random noise, i.e. the binding substance at this location of the substrate did not bind with an analyte, the following steps are carried out. FIGS. 11C and 11E show two possible areas of interest within an evaluation window. First, the surface of the pixels having a high value within the evaluation window of the second matrix is determined and from this surface the radius of the area of interest is determined. Further the radius of this surface is determined from the circumference of this surface. As shown in FIG. 11C, the pixels with a high value do not represent an exact circle, so that the radius determined from the circumference of this area will be greater than the radius of an exact circle 16 shown in FIG. 11C. However, the ratio of the two radii should be less than a predetermined reference value, for example less than 2. FIG. 11D shows all pixels having a high value at the circumference of the surface of the pixels having a high value. The ratio of the radius obtained from the circumference and the radius obtained from the surface is in this case 1.47. This means that an area of interest is present. In FIG. 11F, all pixels having a high value at the circumference of the surface shown in FIG. 11 E are shown and the ratio of the “radius” obtained from the circumference and the radius obtained from the surface shown in FIG. 11E is in this case 4.47. This means that the evaluation window in case of FIG. 11E does not enclose and area of interest, i.e. an area of interest is absent.

As an alternative or as a further step to decide on the presence or absence of an area of interest, it is also possible to take the centre of gravity of a possible area of interest found as the centre of an imaginary circle window having a surface corresponding to the surface of all pixels having a high value within the evaluation window as shown in FIG. 11. Thereafter this imaginary circle window can be moved with respect to the centre of gravity to find a location wherein the imaginary circle window covers a predetermined number of pixels having a high value, for example at least 90% of all pixels. If such a location can not be found, an area of interest is absent. If such a location can be found, an area of interest is present.

The size of the evaluation window used to examine possible areas of interest and to evaluate the signal intensity of an area of interest is determined such that the number of pixels with a high value corresponds with the number of pixels with a low value. This means that in case of presence of a dot area 1, the number of dot pixels and the number of background pixels are equal, i.e. 50% of the total number of pixels. Such a distribution will also be present, i.e. 50% of the pixels within the evaluation window above a mean value and 50% below a mean value, if only noise is present within the evaluation window. Using such an evaluation window shows the advantage that fitting can be performed with maximum reliability in the above described method for evaluation of the signal intensity of the dot area. Finally, if a user wants to move the centre of a dot area or change the size of the evaluation window, the user can do so by means of a suitable user interface.

The invention is not restricted to the above-described embodiments, which can be varied in a number of ways within the scope of the following claims.

1. Method for evaluating the signal intensity of at least one area of a substrate embedded in a substrate surrounding with a background intensity by means of a computer, characterized in that a scatter parameter of the substrate is determined, and in that a matrix (I_{S}) of pixel intensities of pixels located within an evaluation window enclosing the area and surrounding is determined, wherein the pixel intensities of the matrix (I_{S}) are used to obtain an evaluation histogram of pixel intensities, said evaluation histogram showing two distribution peaks, the first distribution peak with the lowest intensity corresponding with the surrounding pixels and the second distribution peak with the highest intensity corresponding with the area pixels, wherein a curve with two peaks is fitted on said evaluation histogram, wherein the scatter parameter is used to correct for scattering either the matrix (I_{S}) or the curve, wherein the signal intensity of pixels in the area is determined by means of data obtained from the curve fitted on said evaluation histogram.

2. Method according to claim 1, wherein the matrix (I_{S}) of pixel intensities is first processed with the scatter parameter to determine the deconvolution of the matrix (I_{S}) to obtain a matrix (I_{D}) of non-scattered pixel intensities, wherein the non-scattered pixel intensities are used to obtain the evaluation histogram, wherein a normal distribution curve determined by means of a noise parameter obtained from the pixel intensities of the matrix (I_{S}), is fitted on each of said two distributions, wherein the mean value of the first fitted distribution curve is taken as the mean pixel intensity of the background intensity and the mean value of the second fitted distribution curve is taken as the mean pixel intensity of the area intensity, wherein the difference of the mean values corresponds with the signal intensity of the pixels in the area.

3. Method according to claim 1, wherein a theoretical scatter histogram of pixel intensities is determined using a theoretical area with predetermined signal intensity (I_{A}) and a surrounding with background intensity (I_{B}) located within an evaluation window corresponding with the evaluation window used to determine the values of the matrix (I_{S}), the scatter parameter and a noise parameter obtained from the pixel intensities of the matrix (I_{S}), wherein said evaluation histogram is made of the pixel intensities of the matrix (I_{S}) and the theoretical scatter histogram is fitted on said evaluation histogram by varying at least the background and area intensities of the theoretical scatter histogram, wherein the difference between the area intensity and background intensity of the fitted theoretical histogram corresponds with the signal intensity of the pixels in the area to be evaluated.

4. Method according to claim 3, wherein a table of normalized scatter response curves is made using a normalized theoretical area, a normalized predetermined intensity, and surrounding without background intensity, wherein the scatter response curves each are determined along a radius from the centre of said theoretical area upto the edge of said evaluation window for each of a number of scatter parameters for different substrates and substrate conditions, wherein a scatter response curve is selected from the table in accordance with the substrate and substrate condition of the substrate used, wherein the selected scatter response curve is used to determine the pixel intensities used to make a scatter histogram and the noise parameter is convoluted with this scatter histogram to obtain the theoretical scatter histogram.

5. Method according to claim 2, 3 or 4, wherein said noise parameter is the standard deviation of the noise present in the pixel intensities of the matrix (I_{S}), wherein the noise standard deviation is preferably determined by determining distribution of the difference between for example each two neighbouring pixels.

6. Method according to any one of the preceding claims, wherein the scatter parameter is determined by illuminating a side of the substrate in a direction parallel to said surface, wherein the pixel intensities along a line on the surface from said side in said illuminating direction are determined to obtain an exponential scatter decay function.

7. Method according to claim 6, wherein the scatter parameter is corrected for signal intensity originating without scattering.

8. Method according to any one of the preceding claims, wherein scatter parameters of different substrates for different conditions of the substrate are determined and stored, wherein a scatter parameter is selected for use in the evaluation in accordance with the condition of the substrate.

9. Method according to any one of the preceding claims, wherein the substrate has an array of areas, wherein a matrix of pixel intensities is determined for each area and corresponding surrounding.

10. Method according to claim 9, wherein the complete surface of the substrate is evaluated to locate the centre of each area and to determine the size of the evaluation window, wherein the location of each area can be corrected through a user interface and thereafter the signal intensity of each area is evaluated.

11. Method for locating possible areas of interest of a substrate embedded in a substrate surrounding by means of a computer, characterized in that an image file of the substrate is processed in a low pass filter algorithm to determine a matrix of local mean values for all pixels of the image file, wherein the matrix of local mean values is combined with the matrix of actual pixel values of the image file to obtain a first matrix of high/low pixel values which are either high or low depending on the actual pixel value being above or below the corresponding local mean value, wherein the high/low pixel values of the first matrix are further processed in a median filter algorithm to obtain a second matrix of median pixel values, wherein each median pixel value equals the majority of the high/low pixel values of the first matrix, wherein the median pixel values are processed row by row and column by column to determine the mean row values and mean column values, respectively, wherein the rows and columns with the highest mean row values and highest mean column values are selected as estimates of the row centre lines and column centre lines of possible areas of interest, the intersections of which are the centres of possible areas of interest.

12. Method according to claim 11, wherein the low pass filter algorithm to determine the matrix of local mean values comprises using a second window with a size enclosing an expected possible area of interest and its surrounding, sliding said second window along the image matrix pixel by pixel and taking, at each position of the second window, the mean value of all pixels within the second window as the local mean value of each pixel at the centre of the second window.

13. Method according to claim 11 or 12, wherein the median filter algorithm comprises comparing the high/low pixel value of each pixel with the high/low pixel values of the surrounding pixels, wherein a high/low pixel value is made equal to the majority of the high/low pixel values of the surrounding pixels.

14. Method according to claim 11, 12 or 13, wherein for each centre of a possible area of interest found a centre of gravity is determined from the median pixel values having a value high within the second window, wherein the centre of gravity is taken as the centre of a possible area of interest.

15. Method according to claim 14, wherein for each centre of a possible area of interest the radius of the possible area of interest is determined from the surface of the median pixel values having a value high and from the circumference of this surface, wherein the ratio of the two radii is determined to decide on the presence or absence of an area of interest.

16. Method according to claim 14 or 15, wherein the centre of gravity of a possible area of interest found is taken as the centre of an imaginary circle window having a surface corresponding to the surface of the median pixel values having a high value, wherein the imaginary circle window is moved with respect to the centre of gravity to find a location covering a predetermined number of the pixels having a high value, wherein the decision on presence or absence of an area of interest is taken in dependence on whether or not such a location is found.

17. Method according to any of claims 11-16, wherein the size of an evaluation window for evaluating an area of interest is determined such that the number of pixels with high value corresponds with the number of pixels with low value.

18. A computer program comprising computer program code means adapted to perform the method of any one of the preceding claims when said program is run on a computer.

19. A computer program as claimed in claim 17 embodied on a computer readable medium or in a file download able in a computer.
